(19) United States
(12) Patent Application Publication
(10) Pub. No.: US 2007/0149981 A1
(43) Pub. Date: Jun. 28, 2007

(54) IMPLANT INSERTION DEVICE

(75) Inventor: Timothy Bhattacharyya, Brookline, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: MASSACHUSETTS GENERAL HOSPITAL, Charlestown, MA (US)

(21) Appl. No.: 11/164,896

(22) Filed: Dec. 9, 2005

(51) Int. Cl. A61F 2/00

(52) U.S. Cl. 606/99

(57) ABSTRACT

Methods and devices are provided for measuring and/or controlling an amount of force applied to an implant or other element. In certain exemplary embodiments, an implant inserter tool is provided that includes a shaft having a proximal end that is adapted to receive a force and a distal end that is adapted to contact an implant. The inserter tool also include a force controlling element coupled to the shaft. In use, the distal end of the tool can be placed in contact with an implant or other element, and a force can be applied to the proximal end of the tool to drive the implant or other element into bone. The force controlling element can measure the amount of force applied to the inserter tool, thereby measuring the force applied to the implant or other element.

### FIELD OF THE INVENTION

The present invention relates to methods and devices for inserting an implant, and in particular to methods and devices for measuring and/or controlling an amount of force applied to an implant or other element during insertion.

### BACKGROUND OF THE INVENTION

Many patients have enjoyed the benefits of joint replacement surgery where an artificial joint is substituted for a degenerate or damaged biological joint. This type of surgery is particularly prevalent in the hip joint, where often the preoperative patient experiences substantial pain in even the routine task of walking. The hip joint replacement operation is typical of joint replacement operations in that the existing joint is removed and a hip replacement system including a femoral component and an acetabular cup (together with a friction-resistant insert) are substituted. In particular, before the surgeon can begin the process of implanting the replacement components, he or she must first make a posteriorlateral incision, retract or dissect the covering musculature, dislocate the hip, and remove the femoral head. The acetabulum must also be reamed out to receive the acetabular cup, and the femur drilled and reamed to receive the femoral component. Once the femur cavity is sufficiently prepared, the surgeon can then insert the femoral component of the prosthesis by applying a force to the femoral component to wedge it into the cavity. Typically this force is applied by hammering the prosthesis into the femur cavity. The surgeon determines the amount of force to apply based upon experience and tactile feedback.

While tactile feedback can be effective, it can sometimes result in the application of too little or too much force to the femoral component. If too little force is applied to the femoral component, the femoral component will not be fully implanted within the femoral cavity potentially resulting in movement of the femoral component which can cause the patient pain. If too great a force is applied to the femoral component, the femur can fracture. When a fracture occurs wider surgical exposure is required to fix the fracture, rendering increased pain and recovery time for the patient.

Accordingly, there is a need for improved methods and devices for measuring and/or controlling an amount of force applied to an implant or other element.

### BRIEF SUMMARY OF THE INVENTION

The present invention provides methods and devices for measuring and/or controlling an amount of force applied to an implant or other element. In one embodiment, an implant inserter tool is provided that includes a shaft having a proximal end that is adapted to receive a force and a distal end that is adapted to contact an implant. The inserter tool also includes a force controlling element coupled to the shaft. In use, the distal end of the tool can be placed in contact with an implant or other element, and a force can be applied to the proximal end of the tool to drive the implant or other element into bone. The force controlling element can measure the amount of force applied to the inserter tool, thereby measuring the force applied to the implant or other element.

The force controlling element can have a variety of configurations. In one embodiment, the force controlling element can be a sensor, such as a piezoelectric sensor. In another embodiment, the force controlling element can be a dampening element such as a piston, a spring, or a compressible member. In yet another embodiment, the force controlling element can be adapted to collapse upon the application of a threshold level of force. For example, the force controlling element can be a torque limiter that is configured to collapse upon application of a threshold level of force thereto to prevent an excess amount of force from being applied to the implant or other element being impacted.

In another embodiment, the inserter tool can be part of a system that includes a driver that can apply force to the inserter tool, and means for controlling an amount of force applied to an implant being inserted into bone using the inserter and driver. The means for controlling an amount of force can be, for example, a sensor, a dampening element, or a torque limiter. The system can also include a variety of other features. For example, the system can include a processor that is adapted to receive patient data and to calculate a threshold force, based on the patient data, that can be applied to an implant without fracturing bone. The system can also optionally include a display element that is adapted to display an amount of force applied to the implant to provide real-time monitoring thereof.

Methods for inserting an implant are also disclosed herein, and in one embodiment, an implant can be positioned adjacent to bone, and an inserter tool can be positioned in contact with the implant. A force can be applied to the inserter tool to drive the implant into bone, and a measurement device coupled to the inserter tool or the implant can provide feedback relating to an amount of force applied to the inserter tool, and consequently the implant. The amount of force can be displayed to provide real-time monitoring.

### BRIEF DESCRIPTION OF THE DRAWINGS

The invention will be more fully understood from the following detailed description taken in conjunction with the accompanying drawings, in which:

FIG. 1 is a perspective view of one exemplary embodiment of an inserter that includes a sensor;

FIG. 2 is a perspective view of an exemplary embodiment of an implant insertion system that includes an inserter having a dampening element and an implant;

FIG. 3A is a perspective view of another exemplary embodiment of an implant insertion system that includes an inserter after the application of a predetermined level of force; and

FIG. 3B is a perspective view of the device of FIG. 3A after the application of an amount of force that exceeds the predetermined level.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

In general, methods and devices are provided for measuring and/or controlling an amount of force applied to an implant, driver, broach, rasp, bone, or other elements. For example, the methods and devices disclosed herein can be used to measure and/or control the forces applied to an implant being driven into bone, thereby reducing or eliminating the risk of fracture to the bone. In certain exemplary embodiments, the various methods and devices can be incorporated into an inserter tool, an implant, a driver, or other devices that are used in procedures which require the application of force. A person skilled in the art will appreciate that, while the present invention is described in connection with driving implants into bone, the methods and devices can be used in a variety of medical procedures and for applying force to a variety of objects.

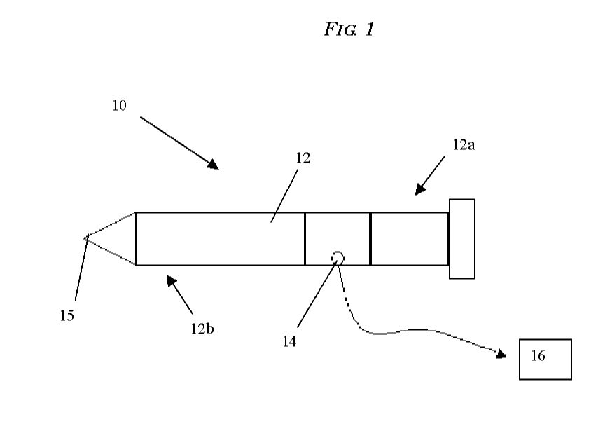

FIG. 1 illustrates one embodiment of an inserter tool 10 that can be used to measure and/or control force applied thereto. This force can be used, for example, to determine the amount of force transferred from the inserter tool 10 to an implant being driven into bone. As shown, the inserter tool 10 generally includes a shaft 12 having proximal and distal ends 12a, 12b, and a force controlling element 14 coupled thereto or formed thereon. The shaft 12 can have virtually any shape or size, but in the illustrated exemplary embodiment it has a generally elongate rigid shape. The proximal end of the shaft 12a can vary in shape and size, but it is preferably adapted to receive a force from a driver, such as a hammer or mallet. In the illustrated embodiment, the proximal end of the shaft 12a includes a flange 17 formed thereon with a planar surface for receiving a force. The distal end of the shaft 12b can also vary in shape and size, and the particular configuration can vary depending upon the intended use. In the embodiment shown in FIG. 1, the distal end 12b is configured to contact an implant to drive the implant into bone. Thus, the distal end 12b has a pointed tip 15 that is configured to fit within a corresponding bore formed in an implant. In other embodiments, the distal end can be round, flat, or have various other geometries to complement the shape of an implant or other element.

As previously mentioned, the inserter tool 10 can also include a force controlling element 14 for measuring and/or controlling an amount of force applied to the shaft 12 of the inserter tool 10, thereby measuring and/or controlling the amount of force transferred from the inserter tool to an implant being driven into bone. While the force controlling element can have a variety of configurations, in one embodiment, the force controlling element can be a sensor 14. The sensor 14 can be located anywhere on the shaft 12 of the tool 10. As shown in FIG. 1, the sensor 14 is located at a mid-portion of the shaft 12 between the proximal and distal ends 12a, 12b. In particular, the sensor 14 is in the form of a housing that is disposed between and separates the shaft into proximal and distal portions 12a, 12b. This allows the force to be transferred from the proximal portion 12a, through the sensor 14, to the distal portion 12b of the shaft 12. The sensor 14 can be mated to the shaft 12 using a variety of techniques known in the art. For example, the sensor can be mated to the proximal and distal portions of the shaft 12 using an adhesive, welding, or other bonding techniques, or in other embodiments it can be integrally formed with the shaft 12. A person skilled in the art will appreciate that, while FIG. 1 illustrates a sensor 14 that is located at a mid-portion of the shaft 12, the sensor can be located anywhere on the shaft or on other devices used in conjunction with the inserter tool 10 to apply or transfer a force.

While virtually any sensor can be used, one exemplary sensor for use with the present invention is a piezoelectric sensor. One suitable piezoelectric sensor is manufactured by PCB Piezoelectrics of Depew, N.Y. (USA) and sold as model number 208c05. The sensor has a range of about 0.05 Newtons to 5000 Newtons, a sensitivity of about 0.22 mV/N, and a resolution of about 0.022N/rms. Another exemplary sensor for use with the present invention is an accelerometer, which measures the acceleration of the inserter tool as a force is applied to it. When little or no acceleration is measured by the accelerometer after the application of a force, the implant will have been substantially secured in position and application of additional force may lead to fracture of the bone.

The sensor 14 can also be configured to communicate with a display, such as a monitor or a screen, for displaying the force measured by the sensor 14. FIG. 1 illustrates wires 19 extending from the sensor 14 and coupled to an external display 16. The display 16 can include or be coupled to a processor, such as a computer, for converting the signal received from the wires into a signal indicative of the force. The signal can be, for example, a visual signal such as a numerical value, a graph, a chart, or a light, and/or an audible signal. While FIG. 1 illustrates an external display 16, the display can be coupled to or formed on the inserter tool, a driver, an implant, or on other devices used to apply or transfer a force. In other embodiments, the display can be worn by a user, such as in the form of a watch.

In another embodiment, the processor can be configured to analyze the measured force received from the sensor 14. For example, the processor can be configured to receive patient data that can be used to calculate a threshold force, or a range of force, that can be applied to an implant being driven into bone without fracturing the bone. The patient data can include, by way of non-limiting example, bone density and strength, sex, age, shape and size of the implant, and other characteristics that may be relevant to determining the threshold force, or a range of force, that can be applied to an implant to drive the implant into bone without fracturing the bone. The threshold force, or the range of force, that is determined based on the patient data can be compared to the measured force received by the sensor and communicated to the processor. The display can indicate whether the measured force is within the predetermined range of force or below the predetermined threshold force, thereby allowing the user to adjust the amount of force applied to the implant as needed. This is particularly advantageous for minimally invasive procedures, where risk of bone fracture is increased.

In use, the distal end 12b of the inserter tool 10 can be placed in contact with an implant, and a force can applied to a proximal end 12a of the tool 10 using a driver, such as a hammer or mallet, to drive the implant into bone. As the force is transferred through the inserter tool 10, the sensor 14 will emit a signal that corresponds to the amount of force applied to the shaft 12, and thus the amount of force transferred to the implant. The signal can then be converted into a value indicative of the force which can be displayed to the user. As previously described, the measured force can also be compared to a predetermined threshold force or a predetermined range of force to prevent fracture of the bone. The surgeon can adjust the applied force when the measured force exceeds the predetermined threshold force.

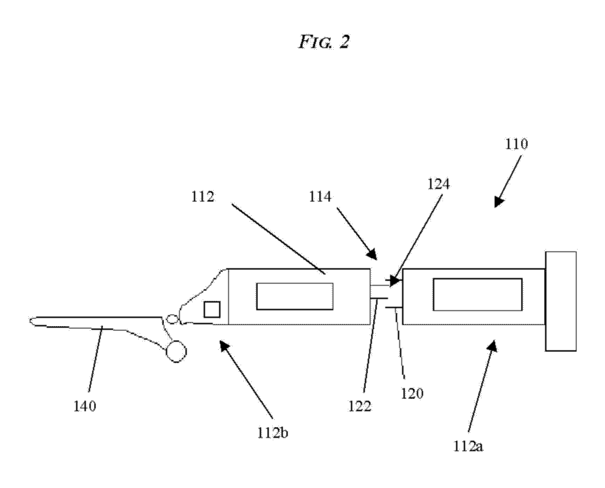

While the force controlling element in FIG. 1 is a sensor, in other embodiments the force controlling element can be a dampening element that is configured to dampen or decrease an amount of force applied to the shaft of the inserter tool, thereby decreasing the amount of force transferred from the inserter tool to an implant being driven into bone. FIG. 2 illustrates one embodiment of an inserter tool 110 having a dampening element 114 formed thereon. In general, the inserter tool 110 includes a shaft 112 that is similar to the shaft discussed in FIG. 1, and a dampening element 114 coupled thereto.

The dampening element 114 can have any configuration, but in an exemplary embodiment it is preferably effective to decrease an amount of force transferred through the shaft 112. As illustrated in FIG. 2, the dampening element 114 is in the form of a piston 114 having a small cylinder 122 that fits within a larger cylinder 120 to displace or compress fluid or air 124 disposed therebetween. A person skilled in the art will appreciate that, while FIG. 2 illustrates a dampening element in the form of a piston 114, the dampening element can have a variety of other configurations. For example, the dampening element can be in the form of a spring, a compressible member, or other structures that absorb or limit force. The dampening element 114 can also include other features to aid in controlling and/or limiting an amount of force transferred from the inserter tool 110 to an implant or other element. For example, the dampening element 114 can be adjustable to allow a user to vary the amount force absorbed by the dampening element 114. Where the dampening element is a piston 114, as shown in FIG. 2, this can be achieved by varying the amount of air or liquid 124 contained between the cylinders 120, 122. Such a configuration will essentially allow a user to select a predetermined threshold force that is applied to an implant 140 or other element being impacted using the inserter tool 110. The dampening element can also include other features, such as a sensor as previously discussed with respect to FIG. 1.

FIG. 2 illustrates the inserter tool 110 in use positioned in contact with a femoral implant 140. The femoral implant 140 can be inserted into a patient's femur by applying a force to the proximal portion of the shaft 112a. As the force is transferred from the proximal portion of the shaft 112a, through the dampening element 114, to the distal portion of the shaft 112b, the dampening element 114 will absorb some of the force. As a result, the amount of force that is transferred to the distal portion of the shaft 112b, and ultimately to the implant 140, is decreased. This is particularly advantageous as the dampening element 114 will prevent too much force from being applied to the implant 140, thereby reducing or preventing the risk of fracture of the bone. As noted above, a user can optionally adjust the threshold force, as may be necessary.

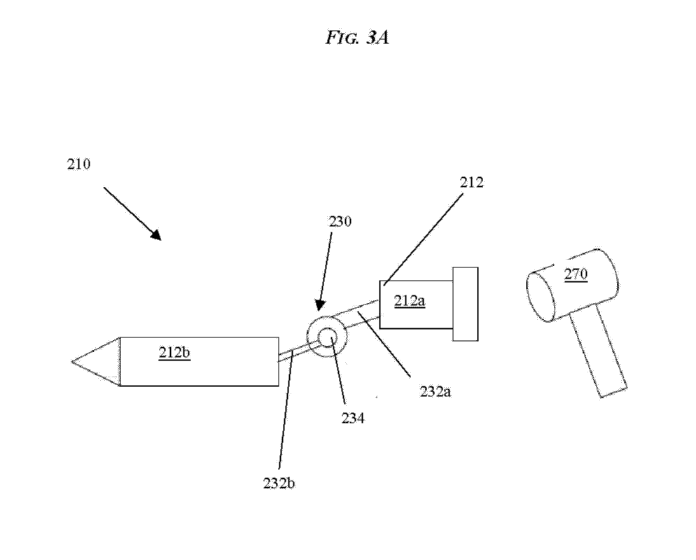

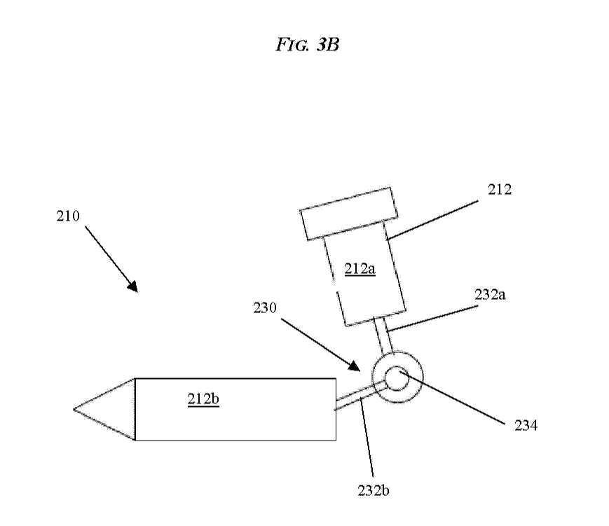

In another embodiment, the force controlling element can be a mechanical torque limiter that allows a portion of the inserter tool to collapse when a threshold force is applied thereto, thereby preventing excess force from being applied to an implant being driven into bone using the inserter tool. FIGS. 3A-3B illustrate one embodiment of an inserter tool 210 having a mechanical torque limiter 214 formed thereon. In general, the inserter tool 210 includes a shaft 212 that is similar to the shaft discussed in FIG. 1, and a mechanical torque limiter 214 coupled thereto between proximal and distal portions of the shaft 212. The torque limiter 214 can have any configuration, but in an exemplary embodiment it is adapted to allow the inserter tool 210 to move from a linear configuration to a non-linear configuration when a threshold force is applied to the inserter tool 210. As shown in FIGS. 3A-3B, the torque limiter 214 has a first arm 232a mated to the proximal portion 212a of the shaft 212, and a second arm 232b mated to the distal portion 212b of the shaft 212. A spring-loaded housing 234 is coupled between the first and second arms 232a, 232b. The spring-loaded housing 234 is adapted to remain in a locked position to hold the arms 232a, 232b, and thus the proximal and distal portions 212a, 212b of the shaft 212, in a linear configuration. When a threshold force is applied to the proximal portion 212a of the inserter tool, the spring-loaded housing 234 will unlock and collapse. As a result, the arms 232a, 232b are free to pivot relative to one another, thereby causing the proximal portion 212a to pivot relative to the distal portion 212b of the shaft 212 such that the proximal and distal portions 212a, 212b are held at an angle with respect to one another. This prevents an excess amount of force from being transferred through the inserter tool 210 to an implant.

A person skilled in the art will appreciate that the torque limiter 214 can also include other features to aid in controlling and/or limiting an amount of force transferred from the inserter tool 210 to an implant or other element. For example, the torque limiter can be adjustable to allow a user to vary the threshold force. While the adjustment techniques can vary, in the embodiment shown in FIGS. 3A-3B the torque limiter 214 can be adjusted by varying the tension of the spring. The mechanical torque limiter can also include a sensor, a processor, a display, and/or a dampening element, as previously discussed with respect to FIGS. 1 and 2.

In use, the distal end of the inserter tool 210 can be placed into contact with an implant (not shown). The force limiter remains in a locked configuration and the threshold force can be set as desired. A driver 270 can be used to apply a force to the proximal end of the shaft 212. As shown in FIG. 3A, when the force is at or below the threshold level, the force is transferred through the shaft 212 and applied to the implant. When the force exceeds the threshold, the torque limiter 214 will unlock and collapse. As a result the proximal portion 212a of the shaft 212 can pivot relative to the distal portion 212b of the shaft 212, as shown in FIG. 3B, to prevent the application of excess force to the implant.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for inserting an implant, comprising:
positioning an implant adjacent to bone;
positioning an inserter tool in contact with the implant; and
applying force to the inserter tool to drive an implant into bone;
wherein a measurement device coupled to the inserter tool provides feedback relating to an amount of force applied to the implant by the inserter tool.

2. The method of claim 1, wherein the feedback from the measurement device is directly proportional to the amount of force applied to the implant.

3. The method of claim 1, further comprising determining a range of force that can be applied to the implant based on patient data.

4. The method of claim 3, wherein the inserter tool limits the amount of force applied to the implant to a force that is within the determined range of force.

5. The method of claim 1, wherein the measurement device is a piezoelectric sensor that emits an amount of current that is directly proportional to the amount of force applied to the implant.

6. The method of claim 1, further comprising dampening the amount of force applied to the implant.

7. The method of claim 6, wherein the amount of force is dampened by a dampening element.

8. The method of claim 7, wherein the dampening element is selected from the group consisting of a spring, a compressible member, and combinations thereof, for dampening the amount of force applied to the implant.

9. The method of claim 1, wherein a portion of the inserter tool collapses upon application of a force that exceeds a threshold level of force.

10. The method of claim 9, wherein the threshold level of force is based on patient data.

11. The method of claim 1, wherein the amount of force is displayed to provide real-time monitoring thereof.

12. An implant insertion system, comprising:
an inserter having a first end for driving an implant into bone and an opposed second end for receiving force;
a driver for applying force to the inserter; and
means for controlling an amount of force applied to an implant being inserted into bone using the inserter and driver.

13. The system of claim 12, wherein the means for controlling an amount force comprises a sensor.

14. The system of claim 12, wherein the means for controlling an amount of force comprises a dampening element.

15. The system of claim 14, wherein the dampening element is selected from the group consisting of a spring, a compressible member, and combinations thereof.

16. The system of claim 12, further comprising a display element adapted to display an amount of force applied to the implant to provide real-time monitoring thereof.

17. The system of claim 12, wherein the means for controlling an amount of force comprises a torque limiter that is adapted to move from a locked position to an unlocked position when a threshold force is applied thereto, the inserter being adapted to move from a linear configuration to a non-linear configuration when the torque limiter is unlocked.

18. An implant inserter tool, comprising:
a shaft having a proximal end adapted to receive a force and a distal end adapted to contact an implant; and
a force controlling element coupled to the shaft between the proximal and distal ends of the shaft, the force controlling element being selected from the group consisting of a sensor, a dampening element, and combinations thereof.

19. The tool of claim 18, wherein the force controlling element comprises a sensor.

20. The tool of claim 18, wherein the force controlling element comprises a dampening element.

21. The tool of claim 20, wherein the dampening element is selected from the group consisting of a spring, a compressible member, and combinations thereof.

22. The tool of claim 18, wherein the device further includes a display element coupled thereto adapted to display an amount of force delivered to the implant to provide real-time monitoring thereof.

23. The tool of claim 18, wherein the shaft includes proximal and distal portions coupled to one another by a torque limiter, the torque limiter being configured to collapse upon application of a threshold level of force thereto such that the proximal and distal portions can be positioned at an angle relative to one another.

24. The tool of claim 18, wherein the torque limiter has first and second arms mated to proximal and distal portions of the shaft and a spring-loaded housing located therebetween, the torque limiter being movable between a locked position in which it maintains the arms in a substantially linear configuration and an unlocked position where it maintains the arms at an angle.

25. The tool of claim 18, wherein distal end has a pointed tip and the proximal end has a head with a planar surface formed thereon.

26. The tool of claim 18, wherein the shaft is rigid.

27. A implant inserter tool, comprising:
a shaft having a proximal end adapted to receive a force and a distal end adapted to contact an implant; and
means for controlling an amount of force applied to an implant being inserted into bone located between the proximal and distal ends of the shaft.

28. The tool of claim 27, wherein the means for controlling force comprises a sensor.

29. The tool of claim 27, wherein the means for controlling force comprises a dampening element.

30. The tool of claim 29, wherein the dampening element is selected from the group consisting of a spring, a compressible member, and combinations thereof.
